(19) United States
(12) Patent Application Publication
(10) Pub. No.: US 2026/0294978 A1
(43) Pub. Date: Oct. 1, 2026

(54) UNIVERSAL TREATMENT OF DOWN SYNDROME

(71) Applicant: DEI Biopharma, Kampala (UG)

(72) Inventor: Matthias Magoola, Kampala (UG)

(21) Appl. No.: 19/096,715

(22) Filed: Apr. 1, 2025

(51) Int. Cl. A61K 35/30; A61K 9/51; A61K 35/28; A61K 35/34; A61K 38/46; A61K 48/00; C12N 5/077; C12N 5/0789; C12N 5/079; C12N 5/0793; C12N 9/22; C12N 15/11; C12N 15/113; C12N 15/86; C12N 15/88; C12N 15/90; C12Q 1/6841; C12Q 1/6883

(52) U.S. Cl. CPC A61K 35/30; A61K 9/5123; A61K 35/28; A61K 35/34; A61K 38/465; A61K 48/005; C12N 5/0619; C12N 5/0622; C12N 5/0647; C12N 5/0657; C12N 9/226; C12N 15/11; C12N 15/1137; C12N 15/86; C12N 15/88; C12N 15/907; C12Q 1/6841; C12Q 1/6883; C12Y 207/07007; C12Y 605/01001; C12N 2310/14; C12N 2310/20; C12N 2506/45; C12N 2510/00; C12N 2750/14143; C12Q 2600/156; C12Q 2600/172

(57) ABSTRACT

Allele-specific (AS) CRISPR-Cas9-mediated cleavage to induce selective loss of the extra chromosome, particularly in a trisomic context where the dosage imbalance is inherently deleterious, can treat Down Syndrome, an untreatable disorder. While it is suitable for individualized therapy, the instant invention provides a generalized treatment module that can be used for any Down Syndrome patient.

### FIELD OF INVENTION

This invention related to gene editing to inactivate or remove an extra chromosome 21 by using a guiding RNA to lead CRISPR-Cas9 to dismantle the said chromosome to treat the untreatable Down Syndrome.

### BACKGROUND OF THE INVENTION

Down syndrome (DS), the most common viable human aneuploidy, arises from an extra copy of chromosome 21(HSA 21 ). Yet, therapeutic strategies to eliminate the supernumerary chromosome in affected cells have been largely undeveloped. A rational therapeutic approach must aim to correct the chromosomal abnormality rather than simply mitigate downstream symptoms. However, chromosome elimination has remained technically challenging due to difficulties targeting a specific homolog among three and the risks associated with non-allele-specific genome editing. A compelling strategy emerges from using allele-specific (AS) CRISPR-Cas9-mediated cleavage to induce selective loss of the extra chromosome, particularly in a trisomic context where the dosage imbalance is inherently deleterious.

The foundation of this argument rests on the feasibility and precision of targeting a single homologous chromosome through haplotype-phased gRNA design. In trisomy 21, where two maternal and one paternal chromosome exist, individual haplotypes exhibit sequence variants that can be exploited to design gRNAs specific to only one allele. Constructing gRNAs that uniquely recognize the M2 maternal allele and validating these through in vitro reporter assays makes it possible to direct Cas9 endonuclease activity to only the supernumerary chromosome. When unrepaired, the resultant DNA double-strand breaks (DSBs) lead to whole chromosome loss. This consequence can be harnessed as a therapeutic mechanism rather than feared as an off-target toxicity.

The efficiency of this approach scales with the number of induced breaks; multiple DSBs across the targeted chromosome significantly increase the likelihood of chromosome loss, as demonstrated by a clear correlation between the number of cleavage sites and trisomy rescue frequency. Importantly, the specificity of allele targeting ensures that only the intended chromosome is eliminated, avoiding the risks of uniparental disomy or damage to essential chromosomal content. Furthermore, by transiently suppressing key DNA repair pathways (LIG4 for NHEJ and POLQ for MMEJ), the cell's ability to rescue cleaved chromosomes is reduced, thereby enhancing the frequency of successful chromosome elimination.

Contrary to alternative methods using allele-nonspecific (ANS) targeting, which simultaneously cleaves all three copies of HSA21 and causes high cellular toxicity and limited karyotype correction, AS targeting shows superior safety and efficacy. This is critical for clinical applications, where the therapeutic index must favor correction over collateral damage. Moreover, the chromosomal correction achieved by AS targeting is functionally meaningful: rescued disomic cells exhibit a reversal of transcriptional dysregulation associated with trisomy 21. Specifically, gene expression profiles shift toward a euploid state, including the upregulation of pathways related to neurogenesis and forebrain development—key deficits in DS—while pathways associated with metabolic stress are downregulated.

An additional strength of this approach lies in its applicability beyond pluripotent stem cells. The elimination of HSA21 using the same AS multi-gRNA vectors in terminally differentiated fibroblasts, including non-dividing cells, demonstrates that the strategy is not limited to early development or regenerative contexts. This broadens the therapeutic scope to include postnatal or even adult interventions, addressing DS as a somatic disorder with a lifelong impact.

Critically, whole-genome sequencing confirms that while small indels and structural variants can arise at target sites, off-target activity is minimal and can be further mitigated through refined gRNA selection. Chromosome elimination does not lead to large-scale genomic instability, as confirmed by G-banding and copy number analysis. The risk of incomplete elimination, leaving a mutated but retained chromosome, is real. However, diminishing this risk is achievable by optimizing cut-site selection, improving delivery efficiency, and transiently impairing DNA repair.

In one embodiment, allele-specific CRISPR-mediated chromosome elimination represents a theoretically sound, experimentally validated, and functionally restorative strategy for trisomic correction in DS. By leveraging the genomic uniqueness of each HSA21 allele in trisomy, this approach introduces a scalable and targeted method to rescue cellular euploidy. Its compatibility with both proliferative and non-proliferative cells and its demonstrated reversal of gene expression abnormalities, position it as a viable candidate for future therapeutic development. The next logical steps are to optimize in vivo delivery mechanisms, evaluate long-term genomic integrity and safety, and expand studies to clinically relevant cell types such as neurons and glial cells—ultimately moving toward therapeutic application in individuals with Down syndrome.

In another embodiment, the method includes using a validated panel of gRNAs that match the most common extra HSA21 haplotypes across populations, with rapid in vitro screening to identify the optimal set for each patient. While the current implementation requires personalized design, the fundamental strategy is generalizable.

### DETAILED DESCRIPTION OF INVENTION

The present invention relates to a therapeutic method for correcting trisomy 21, the genetic basis of Down syndrome, through allele-specific (AS) CRISPR-Cas9-mediated chromosome elimination. The method enables targeted removal of the supernumerary chromosome 21 copy in trisomic cells without affecting the euploid homologs, restoring normal diploid karyotype and reversing gene expression abnormalities associated with Down syndrome.

In one embodiment, the method begins with collecting patient-specific somatic cells, including but not limited to skin-derived fibroblasts or peripheral blood mononuclear cells (PBMCs). These cells are reprogrammed into induced pluripotent stem cells (iPSCs) using non-integrating reprogramming vectors such as Sendai virus, mRNA, or episomal plasmids expressing OCT4, SOX2, KLF4, and c-MYC. Reprogrammed iPSCs are cultured under feeder-free, chemically defined conditions using a medium such as StemFit AK03 on substrates including iMatrix-511. Pluripotency is confirmed by the expression of canonical markers such as OCT4, NANOG, and TRA-1-60, as well as by karyotype and short tandem repeat (STR) profiling.

High-depth whole genome sequencing (WGS) is performed on the trisomic iPSCs, followed by haplotype phasing using custom algorithms and parental sample data (e.g., buccal swabs) to identify the individual HSA21 homologs. This phasing enables the identification of allele-specific single-nucleotide polymorphisms (SNPs) and insertion/deletion (indel) variants unique to one of the three copies, typically the M2 maternal homolog. Computational tools such as Cas-OFFinder identify guide RNA (gRNA) target sequences specific to this homolog, and sequences with ≤2 mismatches to off-target genomic loci are excluded. Between six and thirteen validated gRNAs targeting subtelomeric or interstitial regions of HSA21 are selected. Their specificity and efficacy are confirmed via in vitro EGxxFP plasmid assays co-expressed with Cas9 in a human cell system.

A multicistronic expression plasmid is constructed encoding a high-fidelity Cas9 nuclease variant (eSpCas9(1.1)), the validated set of AS gRNAs, a puromycin resistance gene for selection, and a short hairpin RNA (shRNA) targeting TP53 to enhance survival of edited cells. Cas9 expression is driven by the CAG promoter, and the gRNAs are transcribed under separate U6 RNA polymerase III promoters. The final plasmid is delivered into the patient-derived iPSCs via electroporation using the Neon Transfection System. The electroporation mix includes 16 μg of plasmid, 0.6 μM B18R protein, and optionally, siRNAs targeting DNA repair proteins LIG4 and POLQ (at 0.25 μM each) to suppress nonhomologous end joining (NHEJ) and microhomology-mediated end joining (MMEJ), thereby enhancing the likelihood of complete chromosome elimination.

Following transfection, puromycin (1.0 μg/mL) is applied for three days to enrich the transfected cells. From day five onward, surviving cells are harvested and analyzed for chromosome 21 elimination using fluorescence in situ hybridization (FISH) with BAC probes specific to HSA21 loci (e.g., RP11-15E10, RP11-777J19, RP11-640F21), STR analysis, and loss of fluorescence signals if reporter systems are used. iPSC clones with restored disomy are expanded and validated using G-banding karyotyping and WGS to detect structural variants or off-target effects. RNA sequencing is used to assess the restoration of gene expression, particularly the downregulation of metabolic stress genes and upregulation of genes involved in neurogenesis and forebrain development.

Corrected iPSCs are differentiated into therapeutic cell types, including neurons and glia (for cognitive and developmental correction), cardiomyocytes (to address congenital heart defects), and hematopoietic cells (for immune normalization), using lineage-specific differentiation protocols involving defined signaling modulators. The therapeutic cells are tested in vitro to reverse DS-associated phenotypes, such as mitochondrial dysfunction, proliferation defects, and oxidative stress (ROS). In vivo testing uses xenotransplantation into immunocompromised mice and behavioral assays in transchromosomic mouse models to assess safety, engraftment, and functional recovery.

Genetically corrected cells for clinical use are manufactured under good manufacturing practice (GMP) conditions. This includes whole genome sequencing to confirm genomic integrity, plasmid or vector residue removal, and testing for microbial sterility, mycoplasma, and endotoxins. Cryopreserved, certified therapeutic cell batches are reintroduced into the patient via autologous transplantation routes appropriate to the differentiated cell type, including intrathecal or intracerebral injection for neural precursors and intravenous or intraosseous delivery for hematopoietic cells.

Post-treatment monitoring is performed over short-and long-term intervals to evaluate cognitive performance (e.g., IQ tests, developmental milestones), neuroimaging (MRI, PET), hematologic, cardiac, and immune parameters, and to detect any signs of insertional mutagenesis or re-trisomization.

In an alternative embodiment, the method includes a direct in vivo delivery system, whereby mRNA encoding Cas9 and synthetic gRNAs targeting the specific supernumerary HSA21 allele are formulated into lipid nanoparticles or viral vectors such as adeno-associated virus (AAV) and delivered systemically or locally to target tissues. This embodiment requires efficient tissue-specific delivery, temporally regulated expression of Cas9, and comprehensive safety evaluation.

This method provides a novel, genome-guided therapeutic platform for correcting the fundamental chromosomal defect in Down syndrome by selectively eliminating the supernumerary chromosome 21 copy through allele-specific CRISPR genome editing, enabling a shift from symptomatic management to genetic correction.

### Experimental

A therapeutic method for correcting trisomy 21 in patients with Down syndrome comprises steps beginning with collecting patient-specific somatic cells intended for reprogramming and genome editing. These cells may be obtained through skin biopsy (fibroblasts) or from peripheral blood mononuclear cells (PBMCs) and serve as the starting material for induced pluripotent stem cell (iPSC) generation and/or direct ex vivo genome editing. Reprogramming uses non-integrating vectors such as Sendai virus, mRNA, or episomal plasmids expressing pluripotency transcription factors OCT4, SOX2, KLF4, and c-MYC. The cells are cultured in feeder-free, chemically defined conditions, for example, using StemFit AK03 medium on iMatrix-511 substrate. Successful induction of pluripotency is confirmed via expression of markers such as OCT4, NANOG, and TRA-1-60, alongside karyotype analysis and short tandem repeat (STR) profiling. Whole genome sequencing (WGS) is then conducted at high depth to identify the three distinct chromosome 21 homologs in the trisomic cells. Custom algorithms are applied to phase the alleles by comparing WGS data with parental samples—obtained from buccal swabs—or from derived disomic clones, thereby determining unique single-nucleotide polymorphisms (SNPs) and insertions/deletions (indels) specific to each HSA21 copy for downstream targeting. Using bioinformatics tools such as Cas-OFFinder and in-house phasing pipelines, all candidate Cas9 recognition sequences specific to the target allele (e.g., the M2 maternal homolog) are extracted. Sequences are filtered to exclude those with potential off-targets within two mismatches, and 6 to 13 validated sites are selected across subtelomeric and interstitial regions. Each gRNA target is validated by cloning into EGxxFP reporter plasmids and confirming cutting activity in vitro by co-expressing with Cas9 plasmids. A multicistronic Cas9-gRNA coexpression vector is constructed to deliver the editing machinery into iPSCs. The vector incorporates enhanced-specificity eSpCas9(1.1), 6-13 allele-specific gRNAs, a short hairpin RNA (shRNA) against p53 to improve survival of edited cells, and a puromycin resistance gene for selection. A CAG promoter and the gRNA modules by U6 promoters drive the Cas9 coding region. Transfection of the iPSCs is achieved via electroporation using the Neon Transfection System, with 0.5-1.0×10⁶ cells suspended in 100 μL of Resuspension Buffer R containing 16 μg of the Cas9-gRNA plasmid and 0.6 μM B18R protein, with or without co-transfected siRNAs targeting the DNA repair genes LIG4 (s8179) and POLQ (s21059), each at a final concentration of 0.25 μM. These siRNAs enhance chromosome elimination efficiency by approximately two-fold. After electroporation, puromycin (1.0 μg/mL) is applied for three days to enrich successfully transfected cells. Between days 5 and 10 post-transfection, cells are harvested for downstream analysis. Confirmation of chromosome 21 elimination is performed using fluorescence in situ hybridization (FISH) with HSA21-specific BAC probes (e.g., RP11-15E10, RP11-777J19, and RP11-640F21), STR analysis, and fluorescence signal tracking if a reporter system is employed. iPSC clones that exhibit successful loss of the extra chromosome are isolated and expanded. Disomy is verified via G-banding karyotyping, STR analysis, and whole genome sequencing to rule out structural variants and unintended off-target genome modifications. RNA sequencing assesses gene expression pattern restoration, focusing on neurogenesis markers and metabolic stress.

The corrected iPSC lines are then differentiated into therapeutic cell types suitable for clinical intervention, including neurons and glial cells for cognitive and neurodevelopmental support, cardiomyocytes to address congenital heart defects, and hematopoietic cells to normalize immune function. Differentiation is induced using lineage-specific growth factors and pathway inhibitors. Preclinical testing is conducted in vitro and in vivo to ensure safety and efficacy. This includes evaluation of ROS reduction, cell proliferation, and mitochondrial function, xenotransplantation into immunocompromised mice to assess tumorigenicity and tissue integration, and behavioral rescue studies using transchromosomic mouse models of DS. For clinical use, the corrected cells are manufactured under GMP-compliant conditions. This involves confirmation of genomic integrity via full WGS, complete removal of vector components, and testing for sterility, mycoplasma, and endotoxins. Cells are cryopreserved and certified for therapeutic use. The final therapeutic cells are reintroduced into the patient via autologous transplantation. Neural precursors may be delivered intrathecally or intracerebrally to improve cognitive function, while hematopoietic cells may be infused into the bone marrow to restore immune balance. Patients undergo short-and long-term monitoring following transplantation to evaluate safety, efficacy, and immune response. Assessments include cognitive function tests (e.g., IQ testing, developmental milestones), neuroimaging (MRI, PET), hematologic and cardiac profiling, and genomic surveillance to detect any signs of insertional mutagenesis or re-trisomization.

As an alternative to ex vivo editing, the method also contemplates a direct in vivo editing strategy, which may become feasible with further technology maturation. In this approach, Cas9 mRNA and allele-specific gRNAs would be delivered systemically using lipid nanoparticles or adeno-associated viral (AAV) vectors. For this route to succeed, achieving highly efficient and tissue-specific delivery, tightly controlled Cas9 expression, and broad safety validation across multiple organ systems will be necessary. Altogether, this integrated method offers a comprehensive platform for correcting the genetic defect underlying Down syndrome by selectively eliminating the extra copy of chromosome 21 through allele-specific genome editing, thereby transitioning clinical care from symptomatic treatment toward curative genetic intervention.

The allele-specific CRISPR-Cas9 strategy for trisomy 21 rescue is currently patient-specific because it depends on identifying unique sequence differences—such as single-nucleotide polymorphisms (SNPs) or small insertions/deletions—between the three copies of chromosome 21 in each individual with Down syndrome (DS). These differences are required to design guide RNAs (gRNAs) that selectively target and eliminate the extra chromosome without affecting the other two, thereby avoiding the risk of deleting essential or imprinted chromosomes or inducing uniparental disomy. Since most cases of DS result from nondisjunction during maternal meiosis I, the three homologs are usually genetically distinct, making this specificity feasible but also necessitating whole genome sequencing and haplotype phasing for each patient. However, future developments may make this approach semi-universal or broadly applicable. Large-scale genomic studies could reveal common haplotypes among the extra chromosomes found in most DS patients, allowing pre-designed gRNAs to be used for most cases.

Additionally, strategies targeting unique noncoding repeat regions or exploiting epigenetic differences—such as chromatin accessibility or DNA methylation—could bypass the need for patient-specific sequence variation. Advances in CRISPR technology, including dCas9-based systems or base editors, may further enable chromosome silencing or loss without requiring double-strand breaks, enhancing safety and potentially making treatment more generalizable. A scenario includes using a validated panel of gRNAs that match the most common extra HSA21 haplotypes across populations, with rapid in vitro screening to identify the optimal set for each patient. While the current implementation requires personalized design, the fundamental strategy is generalizable. With improvements in target identification, Cas9 specificity, and delivery methods, it could evolve into a scalable therapy for a wide population of individuals with Down syndrome.

### Expanded Scope of Invention

The present invention relates to therapeutic methods for correcting the chromosomal abnormality associated with Down syndrome (trisomy 21) using allele-specific (AS) CRISPR-Cas9-mediated chromosome elimination. The invention further comprises both patient-specific and non-patient-specific approaches, enabling the targeted removal of the extra copy of human chromosome 21(HSA21 ) from trisomic cells, restoring a normal diploid state, and correcting gene expression abnormalities at their source.

In the patient-specific embodiment, somatic cells (e.g., fibroblasts or PBMCs) are obtained from an individual with trisomy 21 and reprogrammed into induced pluripotent stem cells (iPSCs) using non-integrating vectors expressing OCT4, SOX2, KLF4, and c-MYC. Whole genome sequencing (WGS) and haplotype phasing are performed to distinguish the three HSA21 homologs. Unique single-nucleotide polymorphisms (SNPs) or indels on the supernumerary chromosome are identified, enabling the design of allele-specific guide RNAs (gRNAs). Validated gRNAs are cloned into a multicistronic vector encoding an enhanced-specificity Cas9 variant (eSpCas9(1.1)), a puromycin resistance gene, and a TP53-suppressing shRNA. Delivery into iPSCs is achieved via electroporation, with optional co-transfection of siRNAs targeting LIG4 and POLQ to suppress DNA repair and enhance chromosome elimination. Cells are selected, expanded, validated, and differentiated into therapeutic lineages (e.g., neurons, glia, cardiomyocytes, hematopoietic cells) and administered to the same patient.

In the non-patient-specific (universal) embodiment, the invention includes pre-manufactured, pre-validated gRNA panels that target common SNPs or repeat elements found across the supernumerary HSA21 in most individuals with Down syndrome. Population-scale WGS studies have identified frequent maternal meiotic origin of the third chromosome 21, often involving shared haplotypes or conserved subtelomeric repeats. This invention includes panels of guide RNAs targeting these highly conserved regions of the HSA21 subtelomeric or interstitial genome, particularly those specific to the frequently duplicated maternal homolog. These gRNA panels are validated to have low off-target effects and are incorporated into off-the-shelf plasmid constructs, viral vectors (e.g., AAV), or lipid nanoparticle (LNP) formulations.

In this embodiment, patient genome sequencing and allele phasing are not required. The gRNA-Cas9 systems are delivered directly into patient-derived cells ex vivo or systemically in vivo. The universal vectors contain multiplexed gRNAs (e.g., 6-13) that induce targeted double-strand breaks across the supernumerary chromosome. As in the patient-specific protocol, delivery may be combined with transient suppression of DNA repair pathways using siRNAs or small molecules to enhance elimination efficiency. This platform enables rapid clinical implementation, requiring only karyotypic confirmation of trisomy 21 before treatment.

Furthermore, the invention includes kits and compositions comprising pre-packaged gRNAs, Cas9 proteins or mRNA, delivery vectors, and reagents for cell transfection, selection, and validation. These kits may be used in clinical-grade manufacturing settings under GMP to produce disomic cells ready for therapeutic application. In addition, cell banks may be established from universally corrected iPSC lines that are HLA-matched to populations, enabling allogeneic transplantation of corrected cell types in patients with Down syndrome without requiring custom editing for everyone.

Post-transplantation, patients undergo standardized monitoring protocols to assess therapeutic outcomes, including neurological, cardiac, immune, and developmental function, alongside genome integrity surveillance.

Together, these embodiments provide both customizable and broadly applicable genome editing tools to correct the primary cause of Down syndrome by removing the extra copy of chromosome 21, with the potential for scalable deployment across diverse patient populations.

1. A method for correcting trisomy 21 in a patient with Down syndrome, comprising:
(a) obtaining somatic cells from the patient;
(b) reprogramming said somatic cells into induced pluripotent stem cells (iPSCs);
(c) sequencing the genome of said iPSCs and performing haplotype phasing to identify allele-specific nucleotide sequences unique to the supernumerary chromosome 21;
(d) designing and validating a set of guide RNAs (gRNAs) that target said allele-specific sequences;
(e) constructing a vector expressing Cas9, said gRNAs, a puromycin resistance gene, and a TP53-suppressing shRNA;
(f) transfecting said iPSCs with the vector;
(g) enriching for transfected cells and verifying loss of the targeted chromosome 21 copy;
(h) isolating disomic iPSC clones;
(i) differentiating the corrected iPSCs into therapeutic cell types and
(j) transplanting said therapeutic cells back into the patient.

2. The method of claim 1, wherein the transfection in step (f) further comprises co-delivery of siRNAs targeting DNA ligase 4(LIG4 ) and polymerase theta (POLQ) to suppress double-strand break repair pathways.

3. The method of claim 1, wherein said therapeutic cell types comprise neurons, glial cells, cardiomyocytes, or hematopoietic cells.

4. The method of claim 1, further comprising analyzing the corrected cells by whole genome sequencing, G-banding, RNA sequencing, and fluorescence in situ hybridization (FISH) to confirm correction and absence of structural abnormalities.

5. The method of claim 1, wherein the therapeutic cells are delivered intrathecally, intracerebrally, or via bone marrow infusion.

6. A method according to claim 1, wherein steps (a) through (j) are performed under good manufacturing practice (GMP) conditions for clinical application.

7. A method for in vivo correction of trisomy 21, comprising delivery of Cas9 mRNA and allele-specific gRNAs using lipid nanoparticles or viral vectors to cells within a patient with Down syndrome, wherein said Cas9 and gRNAs selectively induce loss of the supernumerary chromosome 21.

8. The method of claim 1, wherein step (f) further comprises co-delivery of siRNAs targeting DNA ligase 4 (LIG4 ) and polymerase theta (POLQ) to suppress double-strand break repair pathways and increase chromosome elimination efficiency.

9. The method of claim 1, wherein said therapeutic cell types comprise one or more of: neurons, glial cells, cardiomyocytes, or hematopoietic cells.

10. A universal method for correcting trisomy 21 in a patient with Down syndrome, comprising:
(a) selecting from a pre-validated panel of guide RNAs targeting conserved, non-patient-specific genomic sequences on the supernumerary chromosome 21;
(b) delivering said guide RNAs along with Cas9 or Cas9 mRNA into patient-derived cells or tissues using a vector or delivery system selected from the group consisting of: plasmids, viral vectors, and lipid nanoparticles;
(c) inducing targeted double-strand breaks in the supernumerary HSA21;
(d) eliminating the supernumerary chromosome; and
(e) restoring disomy in the patient's cells.

11. The method of claim 10, wherein said delivery system comprises adeno-associated virus (AAV) vectors or lipid nanoparticles encapsulating Cas9 mRNA and synthetic gRNAs.

12. The method of claim 4, wherein steps (a) through (e) are performed ex vivo and the disomic cells are differentiated into therapeutic cell types and transplanted into the patient.

13. A kit for correcting trisomy 21 in a patient with Down syndrome, comprising:
(a) a plurality of validated gRNAs targeting conserved sequences on HSA21;
(b) a Cas9 expression vector or Cas9 mRNA;
(c) delivery components selected from lipid nanoparticles, electroporation buffers, or viral vectors;
(d) reagents for validating chromosome elimination, including FISH probes and STR analysis tools.

14. A composition for the treatment of Down syndrome, comprising a lipid nanoparticle formulation encapsulating Cas9 mRNA and synthetic guide RNAs targeting conserved regions of the supernumerary chromosome 21, formulated for systemic or localized in vivo delivery.
